SEQUENCE LISTING (5 sequences)

SEQ ID NO: 1 - DNA, 255 bp, synthetic construct
  source 1..255: mol_type = other DNA; note = the DNA sequence of the LncRNA CIR6
ttcttggatgacgtcggcgttgctgggagaatgtgccgttcctgccctgcctccacccacctcgggagcagaagcccggcctggacacccctcggcctggacacccctcgaaggagagggcgcttccttgagtaggtgggctccccttgcccttccctccctatcactccatactggggtgggctggaggaggccacaggccagctattgtaaaagctttttattttagtaaaatatacagaagtttgtcttcaa

SEQ ID NO: 2 - DNA, 48 bp, synthetic construct
  source 1..48: mol_type = other DNA; note = forward primer of DNA fragment of the LncRNA CIR6
agttaagcttggtaccgagctcggatccttcttggatgacgtcggcgt

SEQ ID NO: 3 - DNA, 45 bp, synthetic construct
  source 1..45: mol_type = other DNA; note = reverse primer of DNA fragment of the LncRNA CIR6
actgtgctggatatctgcagaattcttgaagacaaacttctgtat

SEQ ID NO: 4 - DNA, 693 bp, synthetic construct
  source 1..693: mol_type = other DNA; note = gene of green fluorescent protein ZsGreen
atggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtaacttccccgccgacggccccgtgatgaagaagatgaccgacaacgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgcgcccagcaagatgcccgactggcacttcatccagcacaggctgacccgcgaggaccgcagcgacgccaagaaccgaagtggcacctgaccgagcacgccatcgcctccggctccgccttgccctga

SEQ ID NO: 5 - DNA, 1076 bp, synthetic construct
  source 1..1076: mol_type = other DNA; note = cTnT promoter sequence
gagtgaaggaaagccttgtgccagcggggccctgaggcgccctgccacagcgcggggcccacagcccaggcggggcccggcggggccgccgcccggcccggcccggccccggccgccgccgccgcgggccggcccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgacagctcctcggcacaggccaggcccctggctctgcccccggagtccgccagcggccggggccagctccctgctgaccgtgggcggcggcagcagggcgcgcccgggcccctctgctcgggccgcagcccggcccggcccgcgccgaggagcccagcgcgcagcagcgggcccggggcagggcggccggagcgcaggcgggccgggccaggccccgcagcgggccagctcctgctgagccgaggctgtgctcccggccgacgccgccgcggggccggcgccgccgccgccgccgccgccgcgggccgccgccgccgccgaggcagctcgagctgagcgcggcgccaggcccaggcccgcgccggcccggcgccgccgaggcgggcagcaggcccggcggcagcaggcccggccccaggccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccg

(19) United States
(12) Patent Application Publication
(10) Pub. No.: US 2025/0041444 A1
(43) Pub. Date: Feb. 6, 2025

(54) Use of Long Non-Coding RNA (lncRNA) in Preparation of Drug for Controlling Myocardial Infarction

(71) Applicant: Yanbian University, Jilin (CN)

(72) Inventors: Lan Hong, Jilin (CN); Xiaotian Cui, Jilin (CN)

(21) Appl. No.: 18/410,402

(22) Filed: Jan. 11, 2024

(30) Foreign Application Priority Data
Aug. 4, 2023 (CN) 2023109737741

(51) Int. Cl. A61K 48/00; A61K 35/28; A61P 9/10; C12N 5/077; C12N 15/86

(52) U.S. Cl. CPC A61K 48/005; A61K 35/28; A61P 9/10; C12N 5/0657; C12N 15/86; C12N 2740/15043; C12N 2750/14143

(57) ABSTRACT

The present disclosure provides use of a long non-coding RNA (lncRNA) in preparation of a drug for controlling myocardial infarction, and belongs to the technical field of biomedicine. The present disclosure further provides use of a recombinant vector with a nucleic acid sequence of lncRNA CIR6 or a recombinant virus strain with the nucleic acid sequence of lncRNA CIR6 in inducing MSC differentiation into a cardiomyocytes in vitro, wherein the MSC is selected from the group consisting of a bone marrow-derived mesenchymal stem cell (BMSC) and an umbilical cord-derived mesenchymal stem cell (UCMSC). In the present disclosure, the lncRNA-CIR6 is derived from human heart. In vivo and in vitro experiments have shown that lncRNA-CIR6 induces the differentiation of the MSC into the cardiomyocyte; meanwhile, the cardiomyocyte detected by flow cytometry has an average transformation rate of not less than 90%.

### CROSS REFERENCE TO RELATED APPLICATION

This patent application claims the benefit and priority of Chinese Patent Application No. 2023109737741, filed with the China National Intellectual Property Administration on Aug. 4, 2023, the disclosure of which is incorporated by reference herein in its entirety as part of the present application.

### REFERENCE TO SEQUENCE LISTING

A Sequence Listing XML file, submitted in accordance with the requirements of 37 C.F.R. §§ 1.831-1.835, entitled “SEQUENCE LISTING.xml,” generated Nov. 30, 2023, and 8,310 bytes in size, has been filed electronically with this application. This Sequence Listing is hereby incorporated by reference into the specification in its entirety.

### TECHNICAL FIELD

The present disclosure belongs to the technical field of biomedicine, and relates to use of a long non-coding RNA (lncRNA) in preparation of a drug for controlling myocardial infarction, methods of treating and/or preventing myocardial infarction using a lncRNA, and a lncRNA, a recombinant vector having a nucleic acid sequence coding for/expressing a lncRNA, or a recombinant virus strain having a nucleic acid sequence coding for/expressing a lncRNA for use in treating and/or preventing myocardial infarction.

### BACKGROUND

Long non-coding RNA (lncRNA) refers to a type of RNA, typically with a length of greater than 200 nucleotides. In contrast to RNA (mRNA) that codes for proteins in the traditional sense, lncRNA does not code for proteins, but regulates gene expression by adsorbing microRNA, supporting proteins, and inhibiting translation. In recent years, not less than 20,000 types of lncRNA have been discovered, which can exert important biological effects by regulating the translation of mRNA and the expression of miRNA, and then participating in the regulation of intracellular molecular networks. Compared with mRNA, lncRNA has diverse sequences, low expression levels, high tissue specificity, and dynamic spatiotemporal expression. However, the vast majority of the not less than 20,000 lncRNAs discovered so far show unclear functions.

Based on current research findings, lncRNA also plays an important regulatory role in cardiovascular diseases such as myocardial infarction, heart failure, and ischemic heart disease. Braveheart (Bvht) is the first lncRNA discovered in mouse heart, and like CARMEN and H19, plays a key role in cardiomyocyte differentiation. Moreover, lncRNA also plays an important role in acute myocardial infarction (AMI). Overexpression of lncRNA HOTAIR improves a cardiac function in diabetic cardiomyopathy, reduces oxidative stress and inflammation, and alleviates cardiomyocyte death. Similarly, lncRNA Gm2691 inhibits the inflammatory response and apoptosis after myocardial infarction, thereby improving cardiac function after AMI. lncRNA MALAT1 can also resist apoptosis to protect cardiomyocytes and improve cardiac function after isoproterenol (ISO) treatment to a certain extent. In addition, lncRNA Snhg1 effectively induces the proliferation of cardiomyocytes through c-Myc, thereby improving cardiac function after myocardial infarction. In contrast, studies have found that lncRNA DACH1 can negatively regulate cardiac regeneration capacity; while silencing lncRNA DACH1 can enhance cardiac proliferation potential, reduce infarct size after ischemic injury, and improve cardiac function. However, there are currently no reports on the induction in regeneration of cardiomyocytes by lncRNA.

Mesenchymal stem cells (MSCs) have strong tissue regeneration ability, low immune rejection, multi-differentiation potential, easy preparation, and large yield, and can be injected intravenously. Accordingly, MSCs transplantation is currently one of the most promising methods for treating myocardial infarction. However, there are urgent needs in regenerative scientific research to regulate the proliferation and differentiation of the MSCs and to complete disease treatment using the MSCs.

### SUMMARY

In view of the foregoing, the intent of the present disclosure is to provide a novel use of a lncRNA for inducing differentiation of a mesenchymal stem cell (MSC), such as a bone marrow-derived mesenchymal stem cell (BMSC) and/or an umbilical cord-derived mesenchymal stem cell (UCMSC), into a cardiomyocyte.

Accordingly, the present disclosure provides a use of a lncRNA, lncRNA CIR6, a recombinant vector including a nucleic acid sequence of lncRNA CIR6, or a recombinant virus strain with a nucleic acid sequence of lncRNA CIR6 for inducing differentiation of an MSC into a cardiomyocyte in vitro,
wherein the MSC is selected from the group consisting of a BMSC and a UCMSC, and
wherein the nucleic acid sequence of lncRNA CIR6 is as set forth in SEQ ID NO:1.

The recombinant vector including the nucleic acid sequence of lncRNA CIR6 may include a backbone vector pCDNA3.1-CMV-MCS-EF1-ZsGreen-T2A-puro.

The nucleic acid sequence of lncRNA CIR6 may be inserted into the BamHI and EcoRI restriction sites within a multiple cloning site (MCS) included in the backbone vector.

The backbone virus for the recombinant virus strain including the nucleic acid sequence of lncRNA CIR6 may be selected from the group consisting of an adeno-associated virus or a lentivirus.

The present disclosure further provides a method for inducing MSC differentiation into a cardiomyocyte in vitro, including the following steps:
introducing lncRNA CIR6, a recombinant vector including the nucleic acid sequence of lncRNA CIR6, or a recombinant virus strain with the nucleic acid sequence of lncRNA CIR6 into an MSC; and
conducting culture to obtain the cardiomyocyte.

The MSC may be selected from the group consisting of a BMSC and a UCMSC.

The nucleic acid sequence of lncRNA CIR6 may be as set forth in SEQ ID NO:1.

The present disclosure further provides a use of lncRNA CIR6, a recombinant vector including the nucleic acid sequence of lncRNA CIR6, or a recombinant virus strain including the nucleic acid sequence of lncRNA CIR6 in preparing a drug for treating and/or preventing myocardial infarction. The nucleic acid sequence of lncRNA CIR6 may be as set forth in SEQ ID NO:1.

The present disclosure further provides use of an MSC in combination with a lncRNA, lncRNA CIR6, a recombinant vector having the nucleic acid sequence of lncRNA CIR6, or a recombinant virus strain having the nucleic acid sequence of lncRNA CIR6 in preparing a drug for treating and/or preventing myocardial infarction. The MSC may be selected from the group consisting of a BMSC and a UCMSC, and the nucleic acid sequence of lncRNA CIR6 may be as set forth in SEQ ID NO:1.

The present disclosure further provides a lncRNA, lncRNA CIR6, a recombinant vector including the nucleic acid sequence of lncRNA CIR6, or a recombinant virus strain including the nucleic acid sequence of lncRNA CIR6. The nucleic acid sequence of lncRNA CIR6 may be as set forth in SEQ ID NO:1. It will be appreciated that the recombinant vector and the recombinant virus strain including the nucleic acid sequence of lncRNA CIR6 may code for/express lncRNA CIR6. Uses for the lncRNA, recombinant vector, and recombinant virus strain of the present disclosure include treating and/or preventing myocardial infarction.

The present disclosure further provides methods for treating and/or preventing myocardial infarction including administering lncRNA CIR6, a recombinant vector having the nucleic acid sequence of lncRNA CIR6, or a recombinant virus strain having the nucleic acid sequence of lncRNA CIR6 as described herein, e.g., to a subject in need thereof. The nucleic acid sequence of lncRNA CIR6 may be as set forth in SEQ ID NO:1.

The present disclosure further provides methods for treating and/or preventing myocardial infarction including administering an MSC in combination with lncRNA CIR6, a recombinant vector having the nucleic acid sequence of lncRNA CIR6, or a recombinant virus strain having the nucleic acid sequence of lncRNA CIR6 as described herein, e.g., to a subject in need thereof. The MSC may be selected from the group consisting of a BMSC and a UCMSC, and the nucleic acid sequence of lncRNA CIR6 may be as set forth in SEQ ID NO:1.

Accordingly, the present disclosure provides use of a lncRNA CIR6, a recombinant vector including the nucleic acid sequence of lncRNA CIR6, or a recombinant virus strain including the nucleic acid sequence of lncRNA CIR6 to induce differentiation of an MSC into a cardiomyocyte in vitro. The MSC may be selected from the group consisting of a BMSC and a UCMSC, and the nucleic acid sequence of lncRNA CIR6 may be as set forth in SEQ ID NO:1. According to the present disclosure, lncRNA-CIR6 may be derived from human heart. Experiments have shown that after lncRNA-CIR6 is introduced into the BMSC or UCMSC to allow culture in vitro, the MSC is induced to transform into the cardiomyocyte detected by immunocytochemical staining. Meanwhile, flow cytometry testing has showed that the cardiomyocyte has an average transformation rate of not less than 90%, proving that the MSC shows significant advantages over other stem cells in an ability to differentiate into the cardiomyocyte. It can be seen that lncRNA CIR6 provided by the present disclosure has a desirable induction effect in inducing the differentiation of BMSCs or UCMSCs into cardiomyocytes. Moreover, based on the efficient induction and differentiation effect of lncRNA CIR6 on specific stem cells, the experiments have verified that this lncRNA provides a new means for stem cell transplantation in treating multiple heart diseases such as myocardial infarction.

### BRIEF DESCRIPTION OF THE DRAWINGS

FIG. 1 shows a map of the pCDNA3.1-CMV-MCS-EF1-ZsGreen-T2A-puro vector;

FIG. 2 shows a transfection flow chart of BMSCs;

FIGS. 3A-3B show an effect of lncRNA-CIR6 on inducing transformation of BMSCs (FIG. 3A) and hUCMSCs (FIG. 3B) into cardiomyocytes (40×);

FIGS. 4A-4B show an efficiency of lncRNA-CIR6 in transforming BMSCs and hUCMSCs into cardiomyocytes, wherein FIG. 4A is a comparison between an empty group and a transfection group of the BMSCs, ****P<0.0001; and FIG. 4B is a comparison between an empty group and a transfection group of the hUCMSCs, ****P<0.0001; notes: data are expressed as means±SEM, n=3;

FIGS. 5A-5B show cardiac function of mice in each group before and after surgery, where FIG. 5A depicts echocardiograms of mice in each group before and after surgery; FIG. 5B depicts histograms obtained by statistically processing cardiac function parameters (LVIDd; LVIDs; LVEF; LVFS) of mice in each group before and after surgery using GraphPad Prism 9.0 software; and

FIGS. 6A-6B show myocardial infarction areas of mice in each group; where FIG. 6A depicts frozen heart sections of mice in each group under a fluorescence microscope; and FIG. 6B depicts histograms obtained by measuring the area of green fluorescence expression in the heart using ImageJ software and then conducting statistical processing of the myocardial infarction area of mice in each group using GraphPad Prism 9.0 software.

### DETAILED DESCRIPTION OF THE EMBODIMENTS

Embodiments of the present disclosure provide use of a lncRNA, lncRNA CIR6, a recombinant vector with a nucleic acid sequence of lncRNA CIR6, or a recombinant virus strain with a nucleic acid sequence of lncRNA CIR6 for inducing differentiation of a mesenchymal stem cell (MSC) into a cardiomyocyte in vitro. In some embodiments, the MSC is selected from the group consisting of a bone marrow-derived mesenchymal stem cell (BMSC) and an umbilical cord-derived mesenchymal stem cell (UCMSC), and in some embodiments, the nucleic acid sequence of lncRNA CIR6 is shown in SEQ ID NO:1 (TTCTTGGATGACGTCGGCGT TGCTGGGAGAATGTGCCGTTCCTGCCCTGCCTCCACCCACCTCGGGAGCAGAAGCCC GGCCTGGACACCCCTCGGCCTGGACACCCCTCGAAGGAGAGGGCGCTTCCTTGAGTA GGTGGGCTCCCCTTGCCCTTCCCTCCCTATCACTCCATACTGGGGTGGGCTGGAGGAG GCCACAGGCCAGCTATTGTAAAAGCTTTTTATTTTAGTAAAATATACAGAAGTTTGTCT TCAA). In some embodiments, the nucleic acid sequence of lncRNA CIR6 is obtained through artificial synthesis.

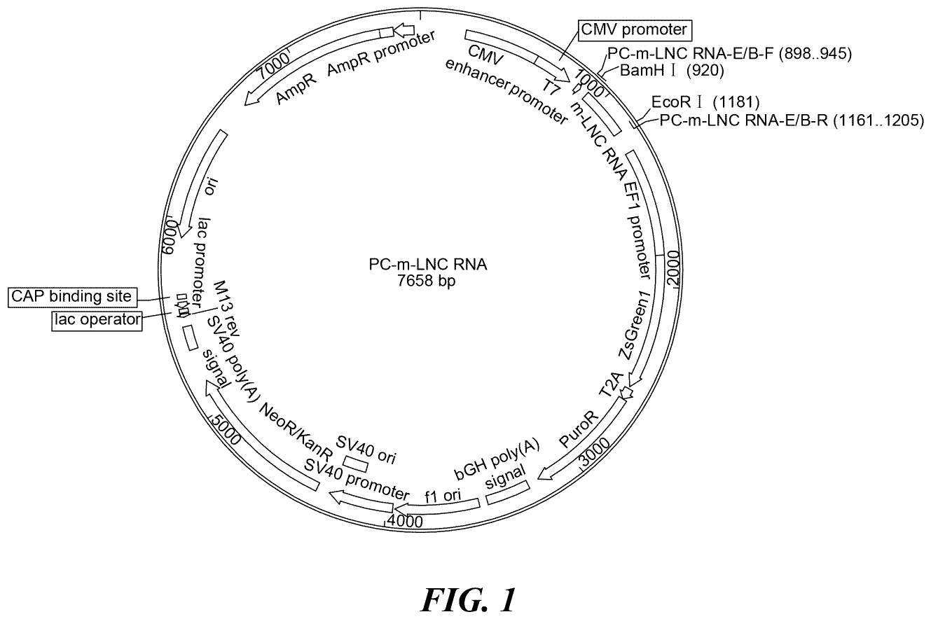

In the present disclosure, the recombinant vector may include the nucleic acid sequence of lncRNA CIR6. In some embodiments, the backbone vector of the recombinant vector includes pCDNA3.1-CMV-MCS-EF1-ZsGreen-T2A-puro, an adeno-associated virus expression vector, or a lentivirus expression vector. In pCDNA3.1-CMV-MCS-EF1-ZsGreen-T2A-puro, CMV and EF1 are gene promoters; MCS is a multiple cloning site; PuroR is a resistance gene fragment, ZsGreen is a green fluorescent protein gene; and T2A is a self-cleaving peptide sequence. In some embodiments, the nucleic acid sequence of lncRNA CIR6 is inserted into the BamHI/EcoRI sites of the MCS in the backbone vector. A map for pCDNA3.1-CMV-MCS-EF1-ZsGreen-T2A-puro is shown in FIG. 1, and was commissioned to be synthesized by Hanbio (Shanghai) Co., Ltd.

In the present disclosure, a construction process of the recombinant vector according to some embodiments may include the following steps:
preparing and/or isolating lncRNA CIR6; and
cloning lncRNA CIR6 into the backbone vector to obtain the recombinant vector.

In some embodiments of the present disclosure, the nucleic acid sequence of lncRNA CIR6 is amplified by PCR using primers to obtain a DNA fragment including the sequence of lncRNA CIR6. The primers may include a forward primer with a nucleotide sequence, e.g., as shown in SEQ ID NO:2 (AGTTAAGCTTGGTACCGAGCTCGGATCCTTCTTGGATG ACGTCGGCGT), and a reverse primer with a nucleotide sequence, e.g., as shown in SEQ ID NO:3 (ACTGTGCTGGATATCTGCAGAATTCTTGAAGACAAACTTCTGTAT). A reaction program of the PCR amplification may include, for example: 95° C. for 5 min; 95° C. for 30 s; 65° C. for 30 s; and 72° C. for (30-60) s/kb, conducted for 27 to 35 cycles; and 72° C. for 10 min. An exemplary reaction system of the PCR amplification is 50 μL, and includes the following components: 25 μL of 2×PCR Buffer, 1 μL of 10 mM dNTPs mix, 2 μL of the forward primer, 2 μL of the reverse primer, 1 μL of a template DNA, 18 μL of ddH₂O, and 1 μL of phanta Super-Fidelity DNA Polymerase.

In the present disclosure, the cloning refers to digesting a backbone plasmid and a target fragment with a same restriction endonuclease, and then ligating a linearized plasmid and the target fragment. There are no special restrictions on the methods of digesting and ligating, and digesting and any ligating methods known to one of skill in the art may be used. After cloning, verification is also conducted. The verification may include: transforming the recombinant vector into an E. coli strain; culturing; and detecting whether the target fragment is present using colony PCR. When the target fragment is present, it is determined that the recombinant vector is successfully constructed and used for subsequent experiments.

In the present disclosure, the recombinant virus strain may include the nucleic acid sequence of lncRNA CIR6. In some embodiments, an initial virus of the recombinant virus strain may include an adeno-associated virus and/or a lentivirus. There is no particular limitation on a construction process of the recombinant virus strain, and any method for preparing the recombinant virus strain known to one of skill in the art can be used.

In the present disclosure, the BMSC or the UCMSC is derived from human beings or rats. Under in vitro conditions, lncRNA CIR6 has an effect of inducing MSC differentiation into cardiomyocytes, and can be used to prepare a biological preparation that induces MSCs to form cardiomyocytes. The experiments have demonstrated that lncRNA CIR6 shows significant differences in ability to induce differentiation of different types of stem cells into cardiomyocytes in vitro. For example, lncRNA CIR6 has a transformation rate of 86% to 96% for inducing differentiation of the BMSCs or UCMSCs into cardiomyocytes, and has a transformation rate of only 70% to 80% for inducing differentiation of human-induced pluripotent stem cells or mouse embryonic stem cells into cardiomyocytes.

The present disclosure further provides a method for inducing MSC differentiation into a cardiomyocyte in vitro, including the following steps:
introducing the recombinant vector in the use into an MSC; and
conducting culture to obtain the cardiomyocyte,
wherein the MSC is selected from the group consisting of a BMSC and a UCMSC.

In the present disclosure, there are no particular restrictions or limitations to conducting culture, i.e., there are no particular restrictions or limitations on isolation and culture methods of the MSCs, e.g., methods for growing/propagating the MSCs in cell culture, and any culture and/or isolation protocols for growing/propagating/isolating MSCs known to one of skill in the art may be used according to embodiments of the present disclosure in order to yield and/or obtain cardiomyocytes differentiated from the MSCs, such as MSCs to which lncRNA CIR6, a recombinant vector including the nucleic acid sequence of lncRNA CIR6, or a recombinant virus strain including the nucleic acid sequence of lncRNA CIR6 have been introduced as described herein, without departing from the scope of the present disclosure.

In the present disclosure, a process for introducing the recombinant vector into the MSC may include: cloning the nucleic acid sequence of lncRNA CIR6 into a plasmid; and transforming the MSC with an obtained recombinant vector. A specific construction process of the recombinant vector can be found in the above description and will not be described in detail here. The transforming may be accomplished using a heat shock method. 6 d after the transformation, a medium is changed. The medium is a cell growth medium. After the transformation is completed, the culturing may be conducted, e.g., for 8 d. The medium is changed every 2 d during the culture.

In the present disclosure, the induced cardiomyocytes are identified using immunocytochemical staining. The results have shown that lncRNA-CIR6 induced rat BMSCs and human UCMSCs (hUCMSCs) to transform into cardiomyocytes. In addition, a transformation effect is also tested. The results show that the efficiencies of lncRNA-CIR6 in converting the BMSCs and hUCMSCs into cardiomyocytes were 86.73%±0.3756 and 95.43±2.130%, respectively. These results show that lncRNA-CIR6 can induce MSCs to transform into cardiomyocytes in vitro, thus providing new avenues for the clinical treatment of diseases related to cardiomyocyte injury.

The present disclosure further provides use of lncRNA CIR6, a recombinant vector with a nucleic acid sequence of lncRNA CIR6, or a recombinant virus strain with the nucleic acid sequence of lncRNA CIR6 in preparation of a drug for preventing and/or treating myocardial infarction, where the nucleic acid sequence of lncRNA CIR6 is as set forth in SEQ ID NO:1. In some embodiments, the recombinant vector and/or the recombinant virus strain with the nucleic acid sequence of lncRNA CIR6 codes for and expresses lncRNA CIR6 in cells, e.g., MSCs, transfected with the recombinant vector and/or the recombinant virus strain.

In an example of the present disclosure, a myocardial infarction mouse disease model is used as a research object. The results show that the cardiac function of the myocardial infarction mouse model transfected with lncRNA-CIR6 and the myocardial infarction mouse model transfected with lncRNA-CIR6 combined with hUCMSCs transplantation is significantly better than that of the myocardial infarction mouse model not transfected with lncRNA-CIR6. This suggests that lncRNA-CIR6 transfection combined with hUCMSCs transplantation can improve the cardiac function of the heart after myocardial infarction and have a protective effect on the infarcted heart.

The present disclosure further provides use of an MSC in combination with the recombinant vector or the recombinant virus strain in the use in preparation of a drug for preventing and/or treating myocardial infarction, where the MSC is selected from the group consisting of a BMSC and a UCMSC; and the nucleic acid sequence of lncRNA CIR6 is as set forth in SEQ ID NO:1.

In the example of the present disclosure, changes in the infarct area of the mouse model before and after administration are also detected. The results show that the post-myocardial infarction area of mice in the lncRNA-CIR6 transfection group and the lncRNA-CIR6 transfection combined with hUCMSCs transplantation group is significantly reduced compared with the myocardial infarction area of the mice in the non-transfected lncRNA-CIR6 group (Control group). Moreover, the myocardial infarction area of mice in the lncRNA-CIR6 transfection combined with hUCMSCs transplantation group is further significantly reduced compared with that of the mice in the lncRNA-CIR6 transfection group alone. This shows that lncRNA-CIR6 transfection combined with hUCMSCs transplantation can reduce the size of myocardial infarction and have a protective effect on the infarcted heart.

The use of a lncRNA in preparation of a drug for preventing and/or treating myocardial infarction provided by the present disclosure will be described in detail below with reference to examples, but they should not be construed as limiting the protection scope of the present disclosure.

### Example 1

Acquisition and Primer Synthesis of lncRNA-CIR6 for Inducing Differentiation of MSCs into Cardiomyocytes

In the present disclosure, according to the prior art (A fetal human heart cardia-inducing RNA (CIR) promotes the differentiation of stem cells into cardiomyocytes, Andrei Kochegarov et al., In Vitro Cell. Dev. Biol.—Animal (2015) 51: 739-748.), a lncRNA was selected from human heart, lncRNA CIR6, which had a nucleotide sequence shown in SEQ ID NO:1.

Based on a nucleic acid sequence of lncRNA-CIR6, amplification primers were designed using Primer3 software. The specific sequences were as follows:

PC-m-LNCRNA-E/B-F:
(SEQ ID NO: 2)
AGTTAAGCTTGGTACCGAGCTCGGATCCTTCTTGGATGACGTCGGCGT;
and
PC-m-LNCRNA-E/B-R:
(SEQ ID NO: 3)
ACTGTGCTGGATATCTGCAGAATTCTTGAAGACAAACTTCTGTAT.

The amplification primers for lncRNA-CIR6 were synthesized by Hanbio (Shanghai) Co., Ltd.

### Example 2

Method for Constructing a Recombinant Vector with lncRNA-CIR6

### 1. Vector Enzyme Digestion

pCDNA3.1-CMV-MCS-EF1-ZsGreen-T2A-puro (designed and packaged by “Hanbio (Shanghai) Co., Ltd.”) was used as a backbone vector. The backbone vector was digested with two restriction endonucleases, BamHI and EcoRI. Each of reagents was added in sequence according to the order in Table 1, mixed gently by pipetting, and reacted in a 37° C. water bath for 1 h to 2 h.

TABLE 1
Linearization reaction system of backbone vector

| Reagent | Volume (μL) |
|---|---|
| Vector DNA (1 μg/μL) | 1 |
| 10 × buffer | 4 |
| ddH₂O | 32 |
| BamHI | 1.5 |
| EcoRI | 1.5 |
| Total | 40 |

2. PCR Amplification of a Target Fragment of lncRNA-CIR6

Hanbio (Shanghai) Co., Ltd. was entrusted to synthesize the nucleic acid sequence of lncRNA-CIR6, a reaction system was prepared according to Table 2, mixed gently, and placed in a PCR instrument to allow a reaction according to the procedure in Table 3.

TABLE 2
PCR amplification system of fragments

| Component name | Volume (μL) |
|---|---|
| 2 × PCR Buffer | 25 |
| dNTPs mix (10 mM each) | 1 |
| F primer (10 μM) | 2 |
| R primer (10 μM) | 2 |
| Template DNA (200 ng/μL) | 1 |
| ddH₂O | 18 |
| phanta Super-Fidelity DNA Polymerase | 1 |
| Total volume | 50 |

TABLE 3
PCR reaction procedure

| Cycle steps | Temperature | Time | Number of cycles |
|---|---|---|---|
| Initial denaturation | 95° C. | 5 min | 1 |
| Denaturation | 95° C. | 30 sec | 27-35 |
| Annealing | 65° C. | 30 sec | |
| Extension | 72° C. | 30-60 sec/kb | |
| Final extension | 72° C. | 10 min | 1 |
| Storage | 12° C. | 0 | 1 |

### 3. Ligation of the Target Fragment to the Vector

A reaction system was prepared in an ice-water bath according to Table 4. The ligation reaction solution was reacted at 50° C. for 30 min, placed on ice for 5 min, and transformed immediately.

### 4. Transformation

DH5 competent cells were taken out of a −80° C. refrigerator and immediately thawed on ice, then aliquoted into each tube with a volume of 50 μL; after aliquoting, the ligation product was added in an amount that did not exceed 1/10 of the competent cell volume, and placed on ice for 20 min to 30 min; heat shock was conducted at 42° C. for 90 s, and the tube was immediately inserted on ice and incubated for 2 min to 3 min after the heat shock; in an ultra-clean bench, 500 μL LB anti-antibody medium was added, and gently inverted up and down 3 to 5 times; the cells were incubated at 37° C. and 230 rpm with shaking for 45 min to 60 min; the bacterial solution was applied to a solid plate with corresponding resistance, spread evenly, and then the plate was placed upside down to allow incubation in a 37° C. incubator for 12 h to 16 h.

TABLE 4
Ligation reaction system

| Component name | Volume (μL) |
|---|---|
| Target gene fragment | 2 (≥100 ng) |
| Linearized vector | 6 (≥50 ng) |
| 2 × HB infusion ™ Master mix | 10 |
| ddH₂O | 2 |
| Total volume | 20 |

### 5. PCR Identification of Bacterial Solution

An identification system was prepared as shown in Table 5, and then PCR amplification of the bacterial solution was conducted as shown in Table 6.

TABLE 5
PCR identification system of bacterial solution

| Component name | Volume (μL) |
|---|---|
| 2 × Hieff PCR Master MIX (Dye) | 5 |
| Primer F | 0.5 |
| Primer R | 0.5 |
| Bacterial solution | 2 |
| ddH₂O | 2 |
| Total volume | 10 |

TABLE 6
PCR identification procedure of bacterial solution

| Cycle steps | Temperature | Time | Number of cycles |
|---|---|---|---|
| Initial denaturation | 94° C. | 5 min | 1 |
| Denaturation | 94° C. | 30 sec | |
| Annealing | 56° C. | 30 sec | 25 |
| Extension | 72° C. | 30-60 sec/kb | 25 |
| Final extension | 72° C. | 10 min | 1 |
| Storage | 12° C. | 0 | 1 |

### 6. Sequencing

Two clones were selected from the screened positive clones for sequencing and comparison. If a sequencing result was consistent with the target sequence, the target plasmid was constructed successfully.

### Example 3

A Method for Inducing Transformation of MSCs into Cardiomyocytes

### 1. Isolation and Culture of MSCs

### 1.1. Isolation and Culture of Rat BMSCs

### 1.1.1. Preparation Before Experiment

(1) 15 SPF-grade SD male rats were selected, weighing 100 g±5 g.
(2) Surgical instruments included: 3 hemostats, 3 ophthalmic scissors, and 3 forceps (sterilized by high-pressure steam before operation).
(3) Complete culture medium: 45 mL of MEM liquid medium was added with 5 mL of fetal bovine serum (FBS) and 500 μL of double antibody; sterile PBS buffer (preheat before use).
(4) Cell culture dish, 15 mL centrifuge tube, 50 mL centrifuge tube, 1 mL syringe, pipette, and pipette tip (all treated by UV irradiation in a clean ultra-bench for 30 min) were selected.

### 1.1.2. Operation Procedures

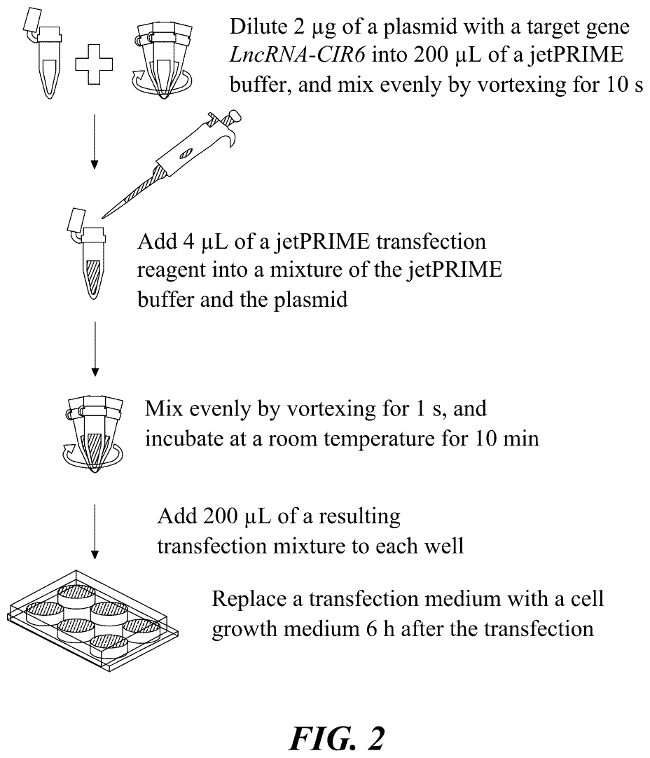

The rats were sacrificed by cervical dislocation and soaked in 75% ethanol for 5 min. The ultraviolet disinfection was conducted on an ultra-clean bench, ventilated with a ventilator for 3 min, and hands were wiped with 75% alcohol. The rats were placed on a petri dish in the ultra-clean bench in a supine position. The rat's hind buttock skin was lifted with hemostats, the skin was cut with ophthalmic scissors, and both femurs were removed under sterile conditions (step 1 in FIG. 2). The muscle tissues attached to the surface of the femur were removed (be careful to avoid damaging the epiphysis) and placed in a 50 mL centrifuge tube filled with 10 mL of sterile PBS. The femur and sterile PBS were poured into a new 10 cm petri dish, epiphyses at both ends were cut off with another pair of ophthalmic scissors (step 2 in FIG. 2), and the medullary cavity was exposed. The complete medium (10 mL) was pipetted into a 50 mL centrifuge tube with a 1 mL syringe to flush the medullary cavity (step 3 in FIG. 2).

### 1.1.3. Cell Preparation

The resulting cell suspension was pipetted to disperse the cells (step 4 in FIG. 2), and centrifuged at 1,000 rpm for 5 min at room temperature (step 5 in FIG. 2). 1.1.4 Inoculation and culture

The resulting supernatant was discarded, and the cells were resuspended in 10 mL of complete medium to prepare a single-cell suspension (step 6 in FIG. 2), the operations were gentle to avoid the generation of air bubbles. The cells were transferred to a 10 cm culture dish and gently shaken (steps 7 and 8 in FIG. 2), and cultured in a cell culture incubator at 37° C. with 5% CO₂ and saturated humidity. The medium was completely changed after 24 h, and then every 2 d. After 7 d, the cell morphology and growth were observed under an inverted microscope. It was seen that the number of adherent cells increased significantly, and cell passage could be conducted when a cell confluence reached 80% to 90%.

### 1.1.5. Cell Passage

The old medium was removed with a pipette pump, and the remaining old medium was washed away with preheated sterile PBS buffer, the above operations were repeated twice, and the washing liquid was discarded. 2 mL of preheated trypsin was added to the cells, the cells were spread evenly quickly, and digested in a cell culture incubator for 2 min to 3 min. After observing under a microscope that 70% to 80% of the cells shrank and became round, an outer wall of the culture dish was slightly tapped to make the cells detach from the surface of the culture dish. 2 mL of the complete medium was added to the clean ultra-bench to terminate the digestion. The cell surface and the bottom of the culture dish were gently pipetted several times with a pipette to dislodge the cells as much as possible and avoid generating bubbles during the operation. The cell suspension was collected in a 15 mL centrifuge tube and centrifuged at 1,000 rpm for 5 min at room temperature. The supernatant was discarded, 1 mL of complete medium was added, and the cells were blown thoroughly, and passaged at 1:2 or 1:3.

1.2. Isolation and Culture of hUCMSCs

### 1.2.1. Preparation Before Recovery

(1) Complete culture medium (45 mL of MEM liquid medium+5 mL of FBS and 500 μL of double antibody) was preheated in a 37° C. water bath in advance.
(2) 15 mL centrifuge tube, cell culture dish, alcohol lamp, pipette, pipette tip (all treated by UV irradiation in a clean ultra-bench for 30 min) were selected.

### 1.2.2. Recovery

(1) 9 mL of preheated complete medium was added into a 15 mL centrifuge tube on a clean ultra-bench.
(2) The frozen cells were placed into disposable gloves, quickly put into a 37° C. water bath and shaken back and forth to allow thawing, and taken out immediately when there was no ice in the cryopreservation tube. The centrifuge tube was sprayed with alcohol and wiped dry, sterilized by heating and opened in the ultra-clean bench, and all cells were transferred to 9 mL of complete medium prepared in advance.
(3) The cells were centrifuged at 1,500 rpm for 5 min at room temperature.
(4) During the centrifugation, 9 mL of complete medium was added from the side wall to the 10 cm cell culture dish in the clean ultra-bench.

### 1.2.3. Cell Inoculation

(1) After the centrifugation was completed, the 15 mL centrifuge tube was taken out, sprayed with alcohol and wiped dry, sterilized by heating and opened in the ultra-clean bench. A resulting supernatant was sucked away with a pipette pump, and 1 mL of complete medium was added and mixed by pipetting, while avoiding the generation of air bubbles during the operations.
(2) 1 mL of cell suspension was added dropwise evenly into the complete medium in the prepared 10 cm culture dish, and shaken evenly with an “8” shape to evenly distribute the cells. The cells were cultured in a cell culture incubator at 37° C. with 5% CO₂ and saturated humidity. The medium was completely changed after 24 h, and then every 2 d. After 7 d, the cell morphology and growth were observed under an inverted microscope. It was seen that the number of adherent cells increased significantly, and the cell passage could be conducted when a cell confluence reached 80% to 90%.

### 1.2.4. Cell Passage

The operations were the same as the passage method of BMSCs.

### 2. Transfection of Recombinant Vector

2.1. 5 μL of the recombinant vector prepared in Example 2 (containing 2 μg of the target gene lncRNA-CIR6) was diluted into 200 μL of jetPRIME buffer, and mixed by vortexing for 10 s.
2.2. The jetPRIME transfection reagent was vortexed for 5 s.
2.3. 4 μL of jetPRIME transfection reagent was added to a mixture of jetPRIME buffer and recombinant vector, and mixed by vortexing for 1 s.
2.4. The mixture was incubated at room temperature for 10 min.
2.5. 200 μL of transfection mixture was added to each well, added dropwise into the cells containing serum medium, and distributed evenly.
2.6. The 6-well plate was gently shaken thoroughly, and then placed in a cell culture incubator at 37° C. with 5% CO₂ and saturated humidity to allow incubation.
2.7. The transfection medium was replaced with a cell growth medium 6 h after the transfection.
2.8. After that, the medium was changed every 2 d, and cells were collected for detection after 8 d of culture.

### 3. Detection of Cardiomyocytes

### 3.1. Immunocytochemistry (ICC) Detection

### 3.1.1. Experimental Materials

### Experimental Group

lncRNA-CIR6 transfection group: the recombinant vector with lncRNA-CIR6 gene fragments was used to transfect BMSCs and hUCMSCs separately.

Empty plasmid transfection group (Vehicle group): an empty vector with only a green fluorescent protein gene fragment was used to transfect BMSCs and hUCMSCs separately.

The antibodies involved were shown in Table 7.

TABLE 7
Types and dosage of antibodies

| Antibody name | Volume (μL) | Dilution factor |
|---|---|---|
| Anti-Cardiac Troponin Tantibody | 0.06 μL | 1:370500 |
| [EPR20266] | | |
| Goat Anti-Rabbit IgG H&L | 120 μL | 1:200 |
| (AlexaFluor ® 647) | | |
| preadsorbed (ab150083) | | |

### 3.1.2. Cell Fixation

The old culture medium was sucked away with a pipette pump, 2 mL of 4% paraformaldehyde was added to each well, fixated at room temperature for 10 min and then discarded, and then the cells were washed 3 times with ice-cold PBS (1 mL per well) for 5 min each time.

### 3.1.3. Cell Permeabilization

The cells were permeabilized with 0.1% PBS-Tween (2 mL per well) for 5 min, then the liquid was discarded, and the cells were washed 3 times with PBS for 5 min each time.

### 3.1.4. Blocking

The cells were blocked with 10% goat serum (2 mL per well) diluted in PBS for 1 h at room temperature.

### 3.1.5. Applying Primary Antibody

The goat serum blocking solution was discarded, the primary antibody was diluted in PBST dissolved in 1% BSA, added to each well at 2 mL, and incubated overnight at 4° C. in a humidified box.

### 3.1.6 Applying Secondary Antibody

The next day, the cells were washed 3 times with PBS for 5 min each time (the next operations were conducted in the dark). The secondary antibody was diluted with 1% BSA and incubated for 1 h at room temperature in the dark. The cells were washed 3 times with PBS for 5 min each time.

### 3.1.7. Nucleus Staining

After the last immersion washing, the PBS was not discarded temporarily to prevent drying of the slides from affecting the staining results. 10 μL of anti-fluorescence quenching mounting solution (containing DAPI) was added dropwise in a center of the adhered slide, a coverslip attached to the 6-well plate was gently picked up with forceps and the water was removed, one side with cells was gently covered on the slide, while avoiding the generation of air bubbles.

### 3.1.8. Image Collection

The slide was placed in a container holder and covered with a light shield. The light source was turned on, the 4× objective lens and the green fluorescence channel were selected. After adjusting the position, brightness, and focus, the expression of green fluorescence was observed and then photographed. The microscope was switched to the red fluorescence channel, the brightness and focus were adjusted, and the expression of red fluorescence was observed and photographed. The microscope was switched to the blue fluorescence channel, the brightness and focus were adjusted, and the cell nucleus was observed and photographed. The operations of the 10× objective lens, 20× objective lens, and 40× objective lens were the same as those of the 4× objective lens.

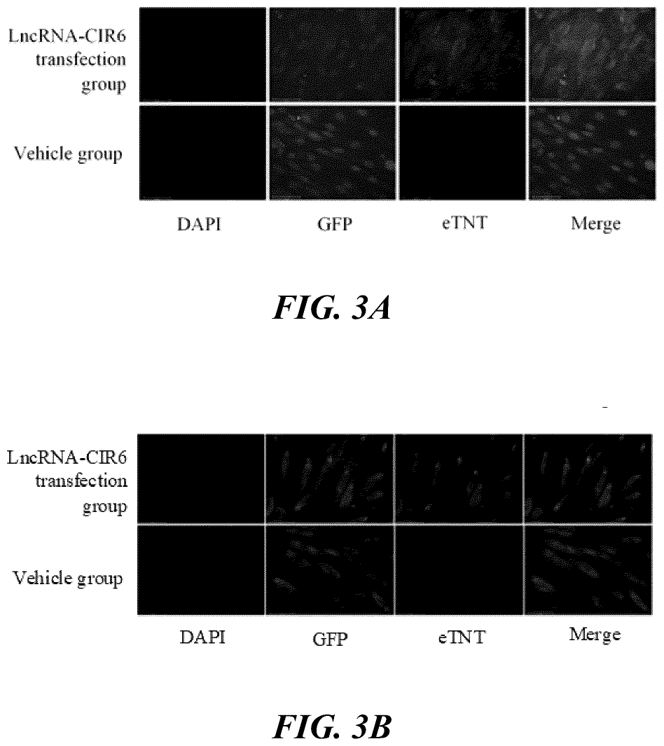

In order to verify whether lncRNA-CIR6 could convert BMSCs and hUCMSCs into cardiomyocytes, an empty plasmid transfection group (Vehicle group) was used as a control group, a myocardium-specific marker cTnT antibody was selected, and fluorescence microscopy was used for observation and analysis. The results showed that after 8 d of induction and culture after transfection with lncRNA-CIR6, both MSCs expressed green fluorescence in the cytoplasm, indicating that lncRNA-CIR6 had been successfully transfected into the cells. At the same time, such cells also clearly expressed cTnT protein marked with red fluorescence, while BMSCs and hUCMSCs transfected with empty plasmid only expressed green fluorescence (FIGS. 3A-B). This suggested that lncRNA-CIR6 had the ability to induce differentiation of both types of MSCs into cardiomyocytes.

### 3.2 Flow Cytometry (Fluorescence-Activated Cell Sorting, FACS) Detection

### 3.2.1. Cell Grouping Solutions and Reagents

(1) Phosphate-buffered saline (PBS) solution
(2) 4% paraformaldehyde
(3) 90% methanol: being cooled before use
(4) Primary antibody: Anti-Cardiac Troponin T antibody [EPR20266], with a dilution of 1:500
(5) Secondary antibody: Goat Anti-Rabbit IgG H&L (Alexa Fluor© 647) spreadsorbed (ab150083), with a dilution of 1:2000
(6) Antibody dilution buffer: 3% BSA aqueous solution by mass percentage

### 3.2.2. Cell Grouping

(1) Group A: BMSCs and hUCMSCs induced and cultured for 8 d after being transfected with the recombinant vector with green fluorescent protein gene fragment and the target gene lncRNA-CIR6.
(2) Group B: BMSCs and hUCMSCs induced for 8 d after being transfected with an empty vector with only a green fluorescent protein gene fragment.
(3) Group C: cells induced and cultured for 8 d after being transfected with lncRNA-CIR6, and then fixated and awaited on-machine detection without primary and secondary antibody treatment.
(4) Group D: cells induced and cultured for 8 d after being transfected with empty vector, and treated with secondary antibodies only.
(5) Group E: normally-cultured BMSCs and hUCMSCs, with only secondary antibody added.

### 3.3. Cell Sample Preparation

(1) Cell fixation: the cells were digested according to the normal passage method, the cell suspension was collected in a 15 mL centrifuge tube, and centrifuged at 300×g for 5 min at room temperature. A supernatant was discarded, the cells were resuspended in 4% paraformaldehyde, mixed well to dissociate and precipitate cells, thus preventing cross-linking of individual cells, and a cell concentration was adjusted to 3×10⁵ cells/mL. The cells were fixated at room temperature for 15 min. The cells in group C were centrifuged and the formaldehyde supernatant was discarded. The cells were resuspended in 1 mL of PBS and stored at 4° C. until detection on-machine.
(2) Cell permeabilization: the cells were centrifuged (300×g, at room temperature for 5 min), 4% paraformaldehyde was discarded, resuspended in pre-cooled 90% methanol, and permeabilized on ice for 15 min.
(3) Applying primary antibody: the cells were separated by centrifugation (300×g, at room temperature for 5 min) and washed with excess PBS to remove methanol, and the operations were repeated twice. The cells were resuspended in 100 μL of diluted primary antibody and incubated for 1 h at 4° C. in the dark. The cells are washed with PBS twice.
(4) Applying secondary antibody: the cells were resuspended in 100 μL of diluted fluorescent substance-conjugated secondary antibody. The cells were incubated at 4° C. for 30 min. The cells are washed with PBS twice. The cells were resuspended in 200 pL to 500 μL of PBS and analyzed on a flow cytometer (all post-staining steps were conducted in the dark).

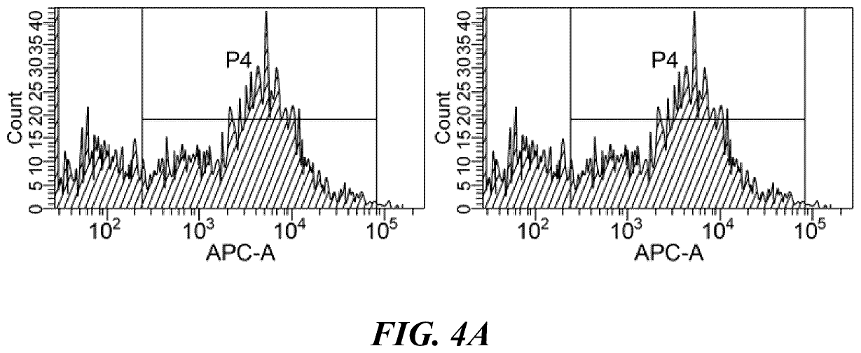

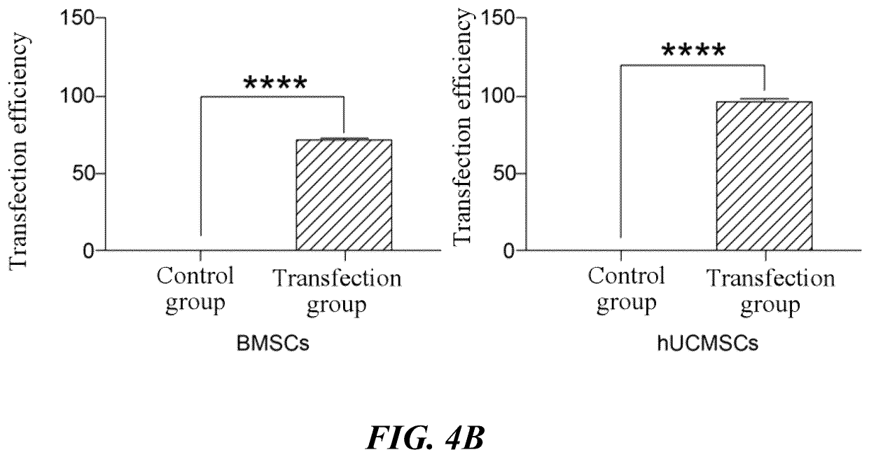

The results were shown in FIGS. 4A-B. lncRNA-CIR6 was transfected into BMSCs (A) and hUCMSCs (B) and then induced and cultured for 8 d. The efficiency of lncRNA-CIR6 in inducing the transformation of two MSCs into cardiomyocytes was measured by measuring an APC fluorescence intensity through flow cytometry. The results showed that both BMSCs and hUCMSCs differentiated into cardiomyocytes. The average transformation rate of lncRNA-CIR6 was 86.73%±0.3756 for BMSCs into cardiomyocytes, and 95.43%±2.130 for hUCMSCs into cardiomyocytes.

### Comparative Example 1

Experiments on the Culture of Embryonic Stem Cells (ESCs) and Induced Pluripotent Stem Cells (iPSCs) and their Transformation into Cardiomyocytes

### I. Isolation and Culture of ESCs

### 1. Preparation Before Recovery:

(1) Matrigel, pre-cooled DMEM/F1 medium, mTesR1 medium, mTesR1 medium containing 10 μM Y-27632, and DMEM/F12 medium were used.
(2) 6-well plate, 15 mL centrifuge tube, cell culture dish, pipette, pipette tip (all treated by UV irradiation in a clean ultra-bench for 30 min) were selected.

### 2. Cell Recovery

(1) The Matrigel was taken out from −80° C. refrigerator, thawed at 4° C., and diluted 100 times with pre-cooled DMEM/F1 culture medium; 1 mL of the Matrigel was added to one well of a 6-well plate, shaken well until it covered the bottom of the well plate, and the 6-well plate was placed in a 37° C. incubator for at least 30 min.
(2) The mTesR1 embryonic stem cell medium was taken out from the 4° C. refrigerator in advance and allowed to stand at room temperature for about 10 min.
(3) The frozen cells were placed into disposable gloves, quickly moved into a 37° C. water bath to thaw, shaken gently until only small pieces of ice crystals remained in the cryopreservation tube, then the cells were taken out, and carefully transferred into a 15 mL centrifuge tube, and mixed well with 5 mL of the mTesR1 medium gently.
(4) The cells were centrifuged at 1,000 rpm for 5 min at room temperature, a supernatant was carefully discarded, the cells were resuspended in 10 mL of mTesR1 medium containing 10 μM Y-27632, inoculated on a Matrigel-coated culture plate, and incubated at 37° C. in an incubator.
(5) After 24 h, the cells were gently washed once with DMEM/F12 medium, transferred into a fresh mTesR1 medium without Y-27632, and cultured in a 37° C. incubator. The medium was completely changed in 24 h, the cells were washed once with preheated DMEM/F12 medium and gently, the DMEM/F12 medium was discarded, a fresh mTesR1 medium was added, and the cells were incubated in a 37° C. incubator until the cell confluence reached 80% to 90% to allow subsequent passage.

### 3. Cell Passage:

When passaging cells, the culture plate was coated with Matrigel in advance. The passaging was conducted at 1:5 to 1:10, the cells were washed 2 times with DMEM/F12 medium, and 1 mL Accutase digestion solution was added to each well of the 6-well plate and digested in a 37° C. incubator for 3 min to 5 min. The cells were observed under an inverted microscope every 2 min, and the digestion solution was carefully discarded when the edges of the clones became bright. 2 mL of mTesR1 medium containing 10 μM Y-27632 was added to gently pipette the cells several times, the cells were collected in a 15 ml centrifuge tube and centrifuged at 1,000 rpm/5 min, and a supernatant was carefully discarded. The cells were resuspended with mTesR1 medium containing 10 μM Y-27632, transferred to Matrigel-coated culture plate, and incubated in a 37° C. incubator. After overnight culture, the cells were transferred to fresh mTesR1 medium without Y-27632 and continued to be cultured in a 37° C. incubator with medium changes every day.

II. Isolation and Culture of iPSCs

### 1. Preparation Before Recovery

(1) STEMCult IPS-XF cell culture medium, DMEM-F12 culture medium, ROCK inhibitor were used.
(2) 15 mL centrifuge tube, cell culture dish, pipette, EP tube, pipette tip (all treated by UV irradiation in a clean ultra-bench for 30 min) were selected.

### 2. Cell Recovery

(1) The Matrigel was taken out from the −80° C. refrigerator in advance, thawed at 4° C., and quickly aliquoted into EP tubes on ice using pre-cooled pipette tip, with each tube of 50 μL to 100 μL, and then stored at 20° C. 50 μL of Matrigel was dissolved in 10 mL of DMEM-F12 and mixed well, 1 mL of the Matrigel was added into a 3.5 mm dish, shaken well, and incubated in an incubator overnight, and the medium was discarded the next day, and washed twice with PBS.
(2) 2 mL of the heated medium was added into a 15 mL centrifuge tube for later use.
(3) The cell cryopreservation tube was taken out from liquid nitrogen, rubbed with hands for a few times, then quickly placed in a 37° C. water bath and shaken quickly. When only small ice crystals were left in the solution in the cryopreservation tube, the tube wall was quickly wiped with alcohol and the tube was transferred to the ultra-clean bench.
(4) The cell suspension was slowly added dropwise into the centrifuge tube prepared in advance, and centrifuged at 1,000 rpm for 3 min.
(5) A supernatant was carefully discarded, a preheated fresh medium was added to gently pipet the cells to be resuspended, the cell suspension was added to a culture dish previously coated with Maitrigel according to a number of cells (10 pM ROCK inhibitor was added), and incubated in an incubator at 37° C. with 5% CO₂, and the medium (without the ROCK inhibitor) was changed the next day.

### 3. Cell Passage:

Cells with a confluence of 80% to 90% were passaged, and a medium containing 10 μL of MROCK inhibitor was prepared, while a KOSR-SerumReplacer fetal calf serum substitute was used to terminate cell digestion.
(1) After aspirating the waste culture medium, the cells were rinsed 2 times with PBS with gentle operations to avoid blowing the cells.
(2) Digestion: 1 mL of 0.5 mM EDTA was added to a 35 mm culture dish, and digested in a 37° C., 5% CO₂ incubator for 3 min to 5 min. When the cell gaps were loose after being observed under a microscope, a medium was added to terminate the digestion.
(3) The culture dish was gently tapped with hands until the cells could completely fall off, and double the amount of KOSR-SerumReplacer fetal bovine serum substitute was added to terminate the digestion.
(4) The cells were centrifuged at 1,000 rpm for 3 min at room temperature.
(5) After discarding the supernatant, 2 mL of fresh medium was added and mixed well, the cells were inoculated in a culture dish coated with Matrigel at a ratio of 1:4, shaken gently thoroughly, and incubated at 37° C. in a 5% CO₂ incubator.
   4. Transfection of ESCs and iPSCs
1. Two new EP tubes were prepared.
2. Tube (1): 5 μL of Lipofectamine™ 3000 was diluted into 125 μL of opti-MEM medium, and mixed well by vortexing for 10 s.
3. Tube (2): 100 μg of the recombinant vector lncRNA-CIR6 prepared in the above example was diluted into 125 μL of opti-MEM medium, and mixed well by vortexing for 10 s. At the same time, an empty plasmid was used to transfect ESCs and iPSCs.
4. The tube (2) was added into tube (1) (at 1:1) and mixed by vortexing for 1 s.
5. The cells were incubated at room temperature for 15 min.
6. 250 μL of transfection mixture was added to each well, added dropwise into the cells in opti-MEM medium, and distributed evenly.
7. The 6-well plate was shaken gently and thoroughly, and incubated in a cell culture incubator at 37° C. with 5% CO₂ and saturated humidity.
8. The medium was changed 6 h after transfection, that is, the opti-MEM medium was replaced with cell growth medium.
9. After that, the medium was changed every 2 d, and cells were collected for detection after 8 d of culture, and the transformation rate was detected by the method in Example 3.

The results showed that lncRNA-CIR6 was transfected into ESCs and iPSCs and then induced and cultured for 8 d. The efficiency of lncRNA-CIR6 in inducing the transformation of two MSCs into cardiomyocytes was measured by measuring an APC fluorescence intensity through flow cytometry. The results showed that both ESCs and iPSCs could differentiate into cardiomyocytes, and lncRNA-CIR6 had an average transformation rate of 70% to 80% for ESCs and iPSCs into cardiomyocytes. In the empty vector treatment group, the transformation efficiencies of ESCs and iPSCs into cardiomyocytes were 9% to 10%, respectively (refer to: A fetal human heart cardiomyocyte-inducing RNA (CIR) promotes the differentiation of stem cells into cardiomyocytes, Andrei Kochegarov et al., In Vitro Cell. Dev. Biol.—Animal (2015) 51:739-748.).

### Example 4

### Experiments on Animal Models of Myocardial Infarction

1. Construction of AAV9 Virus Carrying lncRNA-CIR6 Sequence

A target gene lncRNA-CIR6, a green fluorescent protein ZsGreen (SEQ ID NO:4), and a cTnT promoter sequence (SEQ ID NO:5) were cloned into the AAV9 vector plasmid, and Hanbio (Shanghai) Co., Ltd. was entrusted to construct a recombinant plasmid AAV9-cTnT-lncRNA-CIR6-ZsGreen. The AAV9-cTnT-lncRNA-CIR6-ZsGreen recombinant plasmid was transfected into the HEK293T cell line, while an AAV9 helper plasmid and a packaging plasmid were added to promote the production and packaging of adenovirus. The culture solution was purified and concentrated using methods such as centrifugation to obtain higher-purity AAV9-cTnT-lncRNA-CIR6-ZsGreen virus particles.

### 2. Construction Method of Animal Models of Myocardial Infarction

1.5×10¹² g/mL AAV9 virus carrying the lncRNA-CIR6 sequence was injected into the tail vein of mice at an injection dose of 100 μL, the lncRNA-CIR6 was transfected into the hearts of mice. After 3 weeks, in vivo myocardial infarction models were prepared in mice. Specifically, the mice were placed in an anesthesia box filled with isoflurane (concentration: 5%, flow: 0.5 L/min) to induce initial anesthesia. After righting reflex completely disappeared, the mice were moved to the surface of the operating table and fixed, and the mice were placed supine on a constant-temperature heating pad. With the mouse's head facing the right side of the operator, anesthesia was maintained using a homemade mask supplied with 5% isoflurane. The hair on the neck and left chest area was removed with depilatory cream, and the skin was routinely disinfected with iodophor 3 times. A 0.5 cm long incision was made in the midline of the neck to expose the thyroid gland. The thyroid lobe at the isthmus was separated and the trachea under the sternohyoid muscle was observed. The tongue was moved up to one side of the jaw with forceps by one hand, and then a 20-gauge vessel catheter was inserted into the trachea through the mouth by the other hand. The mice were immediately connected to a ventilator (respiratory frequency 110 times/min, respiratory ratio 1:1, tidal volume 1 mL). At this time, it was seen that the mouse's chest rose and fell uniformly with the frequency of the ventilator, proving that the vascular catheter was correctly inserted into the trachea. At the same time, the isoflurane concentration was adjusted to 2% and the flow rate was reduced to 0.2 L/min. A 1.5 cm long incision was made along the left armpit toward the xiphoid process, the pectoralis major and pectoralis minor muscles were separated, and the chest wall intercostal space was exposed. The third intercostal space was bluntly dissected, and a retractor was used to expand the incision to a width of 6 mm to 8 mm to expose the heart. The pericardium was separated, and the left atrial appendage and left ventricle were fully exposed. Under a magnifying glass, needle ligation was conducted using an 8-0 non-damaging suture needle at 2 mm from a lower edge of the left atrial appendage from left to right. After ligation, it was seen that the left ventricular wall area gradually turned white. The retractor was taken out from the incision, and the third and fourth ribs were sutured with an 8-0 threaded suture needle. When suturing the last time, an outflow tract of the ventilator was blocked for 1 s to 2 s to expand the lungs. The pectoralis major and pectoralis minor muscles were then repositioned, the muscle layer was closed, and the skin incision was sutured. The flow of isoflurane was stopped, until the mouse regained consciousness and breathing, the vessel catheter was slowly pulled out, the respiratory tract was cleared, and the incision was disinfected with iodophor.

1 week after constructing the myocardial infarction model, hUCMSCs were injected into the tail vein (the injection volume was 500,000, specifically dissolved in 100 μL of physiological saline for injection), and the effects of lncRNA-CIR6 transfection and lncRNA-CIR6 transfection combined with hUCMSCs transplantation were observed on cardiac function in mice with myocardial infarction.

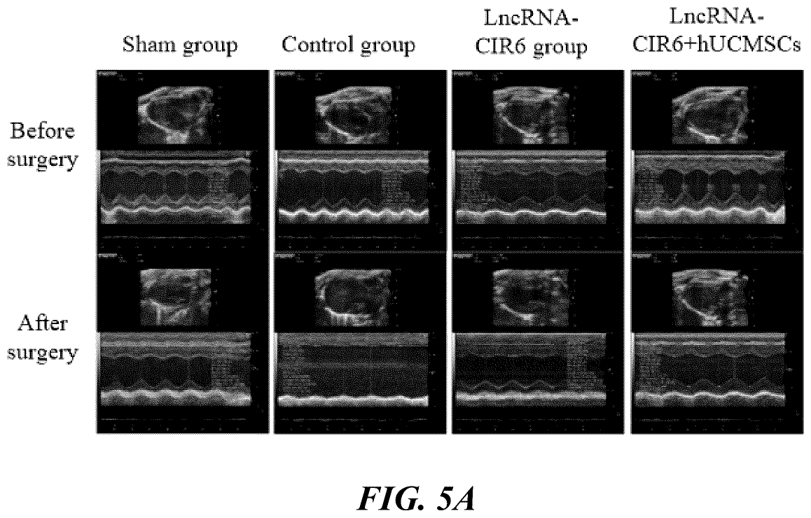

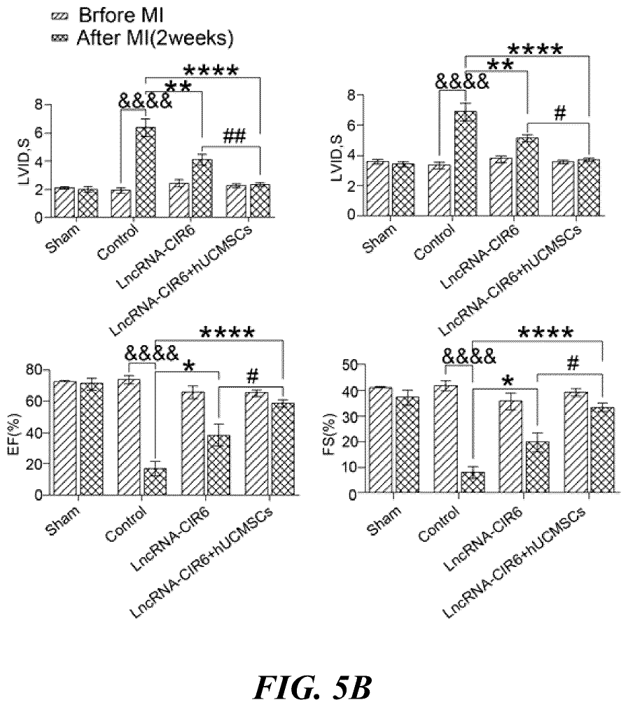

The cardiac function and cardiac function index analysis were measured with a small animal ultrasound instrument. The results in FIGS. 5A-B showed that the left ventricular diastolic and systolic function of mice in the untransfected lncRNA-CIR6 group (Control group) had a strong rhythm before myocardial infarction, and the left ventricular wall was normal. After myocardial infarction, the anterior left ventricular wall became significantly thinner, segmental motion weakened or disappeared, and diastolic function decreased. In the mice of the lncRNA-CIR6 transfection group and the lncRNA-CIR6 transfection combined with hUCMSCs transplantation group, the left ventricular dimension at end-systole (LVIDs) and the left ventricular dimension at end-diastole (LVIDd) after myocardial infarction was significantly reduced compared with those of the mice in the Control group after myocardial infarction, while the left ventricular ejection fraction (LVEF) and the left ventricular fractional shortening (LVFS) were significantly increased. Meanwhile, compared with the lncRNA-CIR6 transfection group, mice in the lncRNA-CIR6 transfection combined with hUCMSCs transplantation group had further significant reductions in LVIDs and LVIDd, and more significant increases in LVEF and LVFS. The results showed that the cardiac function of mice transfected with lncRNA-CIR6 and mice transfected with lncRNA-CIR6 combined with hUCMSCs after myocardial infarction was significantly better than that of mice without lncRNA-CIR6 transfection after myocardial infarction. This suggested that lncRNA-CIR6 transfection combined with hUCMSCs transplantation could improve the cardiac function of the heart after myocardial infarction, and had a protective effect on the infarcted heart.

### Example 5

Transfection of lncRNA-CIR6 into the hearts of mice was carried out by tail vein injection of AAV9 virus carrying the lncRNA-CIR6 sequence. A mouse in vivo myocardial infarction model was prepared 3 weeks later, and hUCMSCs were injected into the tail vein 1 week later. One week later, an ultra-high-resolution small animal ultrasound imaging system vevo2100 was used to determine the effects of lncRNA-CIR6 transfection and lncRNA-CIR6 transfection combined with hUCMSCs transplantation on the cardiac function in mice with myocardial infarction (this process took two days). 9 d after intravenous injection of hUCMSCs, the mice were sacrificed, and the myocardial infarction area was measured using ImageJ software.

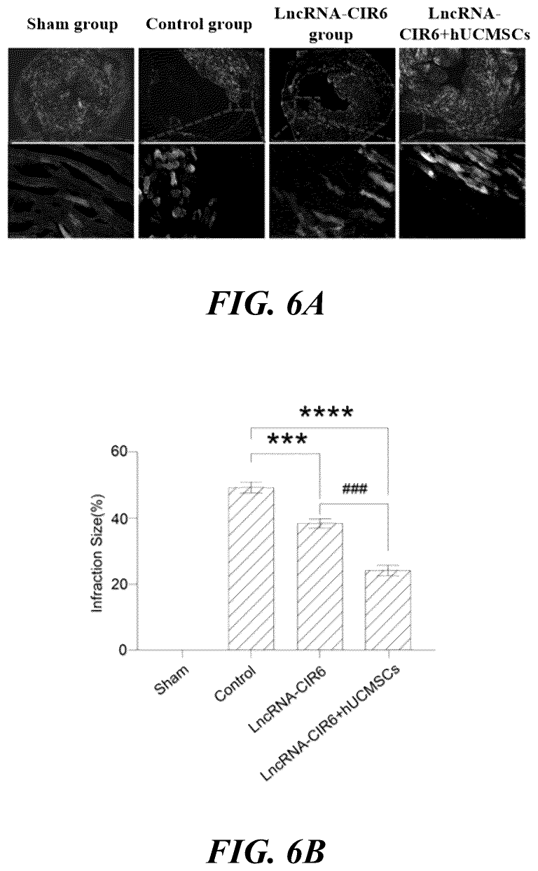

The results were shown in FIGS. 6A-B. The results showed that the myocardial infarction area of mice in the lncRNA-CIR6 transfection group and the lncRNA-CIR6 transfection combined with hUCMSCs transplantation group was significantly reduced compared with the myocardial infarction area of the mice in the non-transfected lncRNA-CIR6 group (Control group). Meanwhile, the myocardial infarction area of mice in the lncRNA-CIR6 transfection combined with hUCMSCs transplantation group was further significantly reduced compared with that of the mice in the lncRNA-CIR6 transfection group alone. This proved that lncRNA-CIR6 transfection combined with hUCMSCs transplantation can reduce the size of myocardial infarction and have a protective effect on the infarcted heart.

The above descriptions are merely exemplary implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for inducing differentiation of a mesenchymal stem cell (MSC) into a cardiomyocyte in vitro, comprising:
introducing a long non-coding RNA (lncRNA) CIR6, a recombinant vector having a nucleic acid sequence of lncRNA CIR6, or a recombinant virus strain having the nucleic acid sequence of lncRNA CIR6 to the MSC; and
culturing the MSC to obtain the cardiomyocyte,
wherein the nucleic acid sequence of lncRNA CIR6 is as set forth in SEQ ID NO:1, and the MSC is selected from the group consisting of a bone marrow-derived mesenchymal stem cell (BMSC) and an umbilical cord-derived mesenchymal stem cell (UCMSC).

2. The method of claim 1, wherein the recombinant vector comprising the nucleic acid sequence of lncRNA CIR6 has a backbone vector pCDNA3.1-CMV-MCS-EF1-ZsGreen-T2A-puro.

3. The method of claim 2, wherein the nucleic acid sequence of lncRNA CIR6 is inserted into BamHI/EcoRI restriction sites within a multiple cloning site (MCS) in the backbone vector.

4. The method of claim 1, wherein a backbone virus of the recombinant virus strain with the nucleic acid sequence of lncRNA CIR6 is selected from the group consisting of an adeno-associated virus or a lentivirus.

5. A lncRNA CIR6, a recombinant vector having a nucleic acid sequence of lncRNA CIR6, or a recombinant virus strain having a nucleic acid sequence of lncRNA CIR6, wherein the nucleic acid sequence of lncRNA CIR6 is as set forth in SEQ ID NO:1.

6. A method for treating and/or preventing myocardial infarction comprising:
administering lncRNA CIR6, a recombinant vector having the nucleic acid sequence of lncRNA CIR6, or a recombinant virus strain having the nucleic acid sequence of lncRNA CIR6 of claim 5 to a subject in need thereof.

7. A method for treating and/or preventing myocardial infarction comprising:
administering an MSC in combination with lncRNA CIR6, a recombinant vector having the nucleic acid sequence of lncRNA CIR6, or a recombinant virus strain having the nucleic acid sequence of lncRNA CIR6 of claim 5 to a subject in need thereof,
wherein the MSC is selected from the group consisting of a BMSC and a UCMSC.

8. A method for treating and/or preventing myocardial infarction comprising:
administering an MSC in combination with lncRNA CIR6, a recombinant vector having the nucleic acid sequence of lncRNA CIR6, or a recombinant virus strain having the nucleic acid sequence of lncRNA CIR6 of claim 5 to a subject in need thereof,
wherein the recombinant vector comprises a backbone vector pCDNA3.1-CMV-MCS-EF1-ZsGreen-T2A-puro.

9. The method for treating and/or preventing myocardial infarction of claim 8, wherein the nucleic acid sequence of lncRNA CIR6 is inserted into BamHI/EcoRI restriction sites within a MCS of the backbone vector.

10. A method for treating and/or preventing myocardial infarction, comprising:
administering an MSC in combination with lncRNA CIR6, a recombinant vector having the nucleic acid sequence of lncRNA CIR6, or a recombinant virus strain having the nucleic acid sequence of lncRNA CIR6 of claim 5 to a subject in need thereof,
wherein the recombinant virus strain comprises a backbone virus selected from the group consisting of an adeno-associated virus and a lentivirus.
